(19) United States
(12) Patent Application Publication
(10) Pub. No.: US 2007/0088318 A1
(43) Pub. Date: Apr. 19, 2007

(54) Calibrated Delivery System for Medical /Dental Implant Surface

(75) Inventors: Matthew J. Allen, Lafayette, NY (US); Kenneth A. Mann, Jamesville, NY (US)

Correspondence Address: BOND, SCHOENECK & KING, PLLC, ONE LINCOLN CENTER, SYRACUSE, NY, 13202-1355, US

(73) Assignee: THE RESEARCH FOUNDATION OF STATE UNIVERSITY OF New York, Albany, NY (US)

(21) Appl. No.: 11/539,472

(22) Filed: Oct. 6, 2006

Related U.S. Application Data
(60) Provisional application No. 60/596,638, filed on Oct. 7, 2005.

(51) Int. Cl. A61M 31/00

(52) U.S. Cl. 604/500

(57) ABSTRACT

A system of delivering a closely controlled volume of a solution carrying a bioactive agent and depositing the solution on a surface of a medical/dental implant in preparation for placing the implant in a patient. The system includes one or more dispensers (cylinders) each terminating in a nozzle, and a plunger (piston) for each dispenser. Each dispenser is filled with the desired volume of solution by common movement of the plungers in an upward direction while the dispenser nozzles are connected to an adapter unit having a single nozzle immersed in the solution. The dispenser nozzles are then removed from the adapter and placed in mating hubs on a domed lid portion of a dosing chamber.

### PRIORITY CLAIM

The present application claims priority to U.S. Provisional Patent Application Ser. No. 60/596,638, filed Oct. 7, 2005, the entirety of which is hereby incorporated by reference.

### BACKGROUND OF THE INVENTION

The present invention relates to medical/dental implant technology, and more specifically to improvements in applying a layer of bioactive agent to a surface of an item in preparation of its implantation into a patient.

Bioactive agents are increasingly used in connection with medical and dental implant procedures. A liquid solution of such agent(s) is applied to at least a portion of the implant prior to inserting the implant in the intended location in the patient. It is highly desirable, and in some cases critical, that the volume of solution applied to the implant be precisely controlled. For example, if the bioactive agent is a growth enhancer, depositing too much of the solution on the implant may result in unwanted bone growth.

The term “bioactive agent” or “biologically active material” encompasses therapeutic agents, such as drugs, and also genetic materials and biological materials.

The genetic materials mean DNA or RNA, including, without limitation, of DNA/RNA encoding a useful protein stated below, anti-sense DNA/RNA, intended to be inserted into a human body including viral vectors and non-viral vectors. Examples of DNA suitable for the present invention include DNA encoding:

(1) anti-sense RNA, tRNA or rRNA to replace defective or deficient endogenous molecules;

(2) angiogenic factors including growth factors, such as acidic and basic fibroblast growth factors, vascular endothelial growth factor, epidermal growth factor, transforming growth factor .alpha. and .beta., platelet-derived endothelial growth factor,

platelet derived growth factor, tumor necrosis factor .alpha., hepatocyte growth factor and insulin like growth factor;

(3) cell cycle inhibitors including CD inhibitors;

(4) thymidine kinase (“TK”) and other agents useful for interfering with cell proliferation; and

(5) the family of bone morphogenic proteins (“BMP's”) as explained below.

Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor .alpha. and .beta., platelet derived endothelial growth factor, platelet derived growth factor, tumor necrosis factor .alpha., hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7.

Alternatively or in addition, molecules capable of inducing an upstream or downstream effect of a BMP can be provided. Such molecules include any of the “hedgehog” proteins, or the DNA's encoding them. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the implant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Biologically active material also includes non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; immunosuppressants such as sirolimus (RAPAMYCIN), tacrolimus, everolimus and dexamethasone, antineoplastic/antiproliferative/anti-miotic agents such as paclitaxel, 5-fluorouracil, cisplatin, vinblastine, vincristine, epothilones, methotrexate, azathioprine, halofuginone, adriamycin, actinomycin and mutamycin; cladribine; endostatin, angiostatin and thymidine kinase inhibitors, and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; bone anabolic factors such as parathyroid hormone and prostaglandin receptor agonists; antiresorptive agents such as bisphosphonates, cathepsin K antagonists and alphav beta3 integrin antagonists.

The principal object of the invention is to provide a system for delivering a precisely controlled volume of liquid/flowable solution to a surface of a medical/dental implant.

A further object is to provide a novel system for delivering closely controlled, small volumes of liquid onto an implant surface.

Another object is to provide a bioactive agent delivery device which is conducive to use under sterile conditions, such as in an operating room.

Other objects are: to provide vacuum assisted delivery of small volumes of fluid to an implant surface, thereby ensuring complete and efficient transfer; to provide a device for delivery of a solution containing a bioactive agent which may be customized to permit precise localization of the solution on an implant; and, to provide a system for delivering a bioactive agent to an implant surface which ensures consistent delivery for all implants.

Other objects will in part be obvious and will in part appear hereinafter.

### SUMMARY OF THE INVENTION

In the disclosed embodiment the calibrated delivery system includes a liquid transfer adapter, a differential dosing device, and a vacuum dosing chamber. Initially, the liquid transfer adapter and a portion of the differential dosing device are interconnected to load the dosing device with the solution of bioactive agent. The liquid transfer adapter is in the form of a flat plate having one or more hubs on one side thereof, and on the other side a single nozzle, the hub(s) and nozzle communicating through passageways internally of the plate. The dosing device includes one or more dispensers (cylinders), the number and arrangement of which is determined by the particular size and shape of the implant with which the delivery system is to be used, and the desired pattern of distribution of the liquid on the implant. Likewise, the number and arrangement of hubs on the transfer adapter corresponds to the number and arrangement of dispensers on the dosing device. The dispensers terminate in individual nozzles which are inserted into the hubs of the adapter and the single nozzle on the lower side of the adapter is inserted into the solution of bioactive agent. A plunger (piston) is positioned within each of the dispensers on the dosing device; preferably, when the number of plungers is greater than one, all plungers will be connected for equal, simultaneous movement within the dispensers. The plungers are at their lowermost position when the nozzle is inserted into the liquid and the plungers are then moved upwardly to draw liquid into the dispensers. Although all plungers are moved the same distance, the amount of liquid drawn into each dispenser may be varied by varying the diameter of the individual nozzles at the lower end of each dispenser.

When the fluid has been drawn into the dispenser(s) the nozzles on the dosing device are removed from the hub(s) of the transfer adapter and placed in opening(s) in a cover or lid portion of the dosing chamber. In addition to the lid, the dosing chamber includes a base portion wherein the implant is supported. The lid and base are configured and dimensioned for mating fit, preferably with a gasket at the mating surfaces. In the disclosed embodiment, the base unit includes a port which is connected to a vacuum pump operable to lower the pressure within the assembled lid and base to somewhat below atmospheric. The solution is injected from the dispenser(s) into respective passageways in the lid by downward movement of the plungers. The vacuum pump is then turned on, drawing out solution from the passageways and ensuring efficient transfer of solution to the implant, the vacuum flow being directed to the perimeter of the implant. When visual inspection (through the transparent lid) confirms adequate transfer of solution on the surface of the implant, the vacuum pump is turned off, the lid is removed from the base and the implant is removed from the device and placed in the patient.

The foregoing and other features of construction and use of the invention will be more easily understood and fully appreciated from the following detailed disclosure, taken in conjunction with the accompanying drawings.

### BRIEF DESCRIPTION OF THE DRAWINGS

FIG. 1 is a front elevational view of two interconnected portions of the device of the invention;

FIG. 2 is a front elevational view of one of the portions of FIG. 1 interconnected to another portion of the device.

FIG. 3 is a front elevational view of the portion of the device shown in both FIGS. 1 and 2 in association with other portions of the device;

FIG. 3A is an enlarged, fragmentary portion of FIG. 3;

FIG. 4 is a side elevational view of portions of the device shown in FIG. 3;

FIG. 5 is a top plan view of the portions shown in FIG. 4;

FIG. 6 is a perspective view of the present invention; and

FIG. 7 is an exploded perspective view of the present invention.

### DETAILED DESCRIPTION

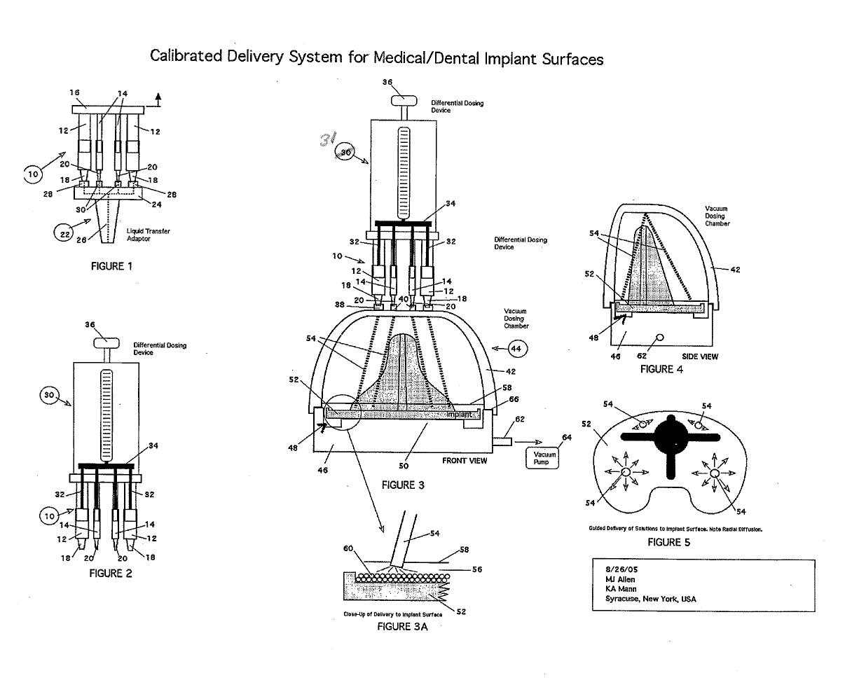

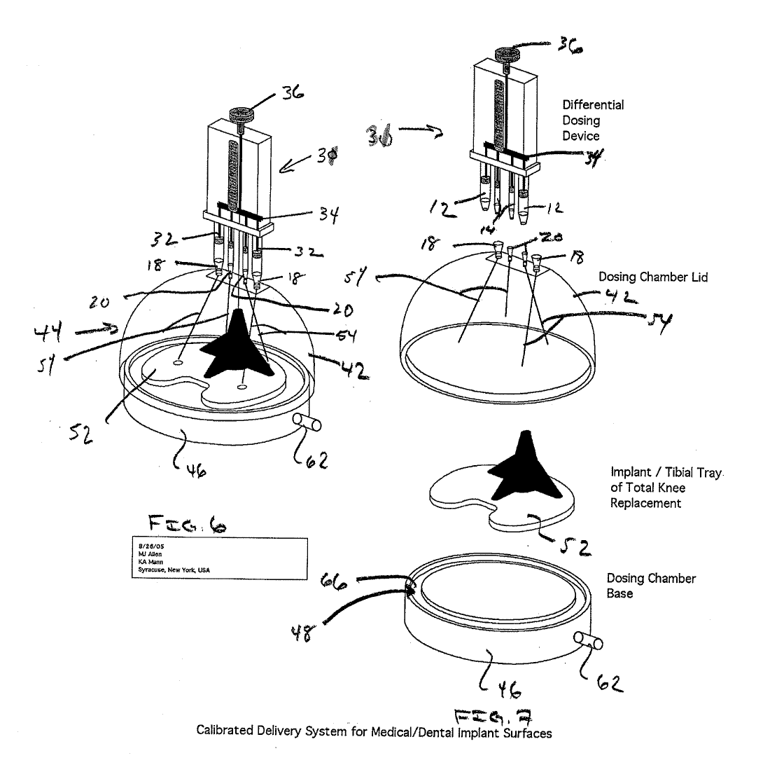

Referring now to the drawings, in FIG. 1 is shown an element termed a variable volume dispenser assembly and denoted by reference numeral 10. Dispenser assembly 10 includes one or more hollow cylinders or dispensers; in the illustrated embodiment a plurality of dispensers are shown, including a first pair 12 of relatively larger diameter, and a second pair 14 of relatively smaller diameter. All four dispensers are joined at their upper ends by plate 16. Dispensers 12 and 14 terminate at their lower ends in nozzles 18 and 20, respectively. A second element, termed a liquid transfer adapter and denoted by reference numeral 22 includes a flat plate 24 having a single nozzle 26 extending from its lower (as seen in FIG. 1) surface. A pair of relatively larger hubs 28 and a pair of relatively smaller hubs 30 extend from the upper surface of plate 24 and surround openings which communicate via internal passageways with the bore of nozzle 26. Dispenser assembly 10 and adapter 22 are shown in FIG. 1 with nozzles 18 inserted in hubs 28 and nozzles 20 inserted in hubs 30. The size and locations of the hubs on the adapter correspond to the size and location of the mating nozzles on the dispenser assembly. That is, the number and size, as well as relative location of dispensers on the assembly may vary with the particular implant with which the device is to be used, and the hubs on the adapter likewise vary to conform to the dispenser nozzles.

Portion 10 of the dosing device is shown in FIG. 2 in association with calibrated dosing gun 31. Individual plungers 32 extend into dispensers 12 and 14, forming pistons at their lower ends which fit closely within the cylinders formed by the dispensers. Plungers 32 are joined at their upper ends by connector 34 for joint, equal, reciprocal, vertical movement in response to movement of handle member 36 to which connector 34 is attached. Handle member 36 may be threaded and/or include a ratchet or detent mechanism for selective, calibrated movement. Upward movement of plungers 32 draws fluid into dispensers 12 and 14 through nozzle 26 and the individual nozzles of the dispensers, the amount of liquid being directly proportional to the distance of movement.

When the dispensers have been filled to the desired extent with the solution of bioactive agent, adapter 22 is removed and nozzles 18 and 20 are inserted into hubs 38 and 40, respectively, of lid portion 42 of dosing chamber 44, as seen in FIG. 3. In addition to lid portion 42, dosing chamber 44 includes base portion 46 having a recess 48 extending around its periphery. The portion surrounded by recess 48 provides a support 50 for the implant 52 upon which the solution is to be deposited. Lid portion 42 is formed of transparent plastic to enable viewing of the implant. Passageways 54 in lid portion 42 communicate at one end withy nozzles 18 and 20 and at the other end with a space, denoted in FIG. 3A by reference numeral 56 between a lower surface 58 of lid 42 and the upper surface of implant 52. Solution 60 is seen in FIG. 3A exiting passageway 54 and deposited on implant 52. Integral port 62 provides means for connecting vacuum pump 64 to the recess within base portion 46. Gasket 66 is preferably provided at the mating peripheries of lid and base portions 42 and 46, respectively, to improve efficiency of the seal when vacuum is applied.

As best seen in FIG. 3A, lower surface 58 of lid portion 42 is immediately above, but spaced a short distance from the upper surface of implant 52. This is important since the implant could be damaged by contact with the lid, and without sufficient space between the two the solution may stick to the lid surface rather than being deposited upon the surface of the implant. The number and size (diameter) of passageways 54 are customized to the desired location of the deposit of solution onto the implant surface. An example of solution distribution is shown in FIG. 5. The diameters of passageways 54 are not necessarily the same. Indeed, since larger passageways can potentially deliver greater volumes of solution, this presents another opportunity for customizing the chamber lid to the particular application. Since various portions of the device may be customized for use with various types, shapes and sizes of implants, indexing means such as a peg on one portion mating with a hole in another may be employed to ensure that the dosing device 10 and lid 42 are compatible.

In practice, the nozzles of the dispensers are inserted into the openings defined by the hubs on the adapter, the adapter nozzle is immersed in the solution to be delivered and the plungers are moved upwardly by a calibrated distance to draw the desired amount of solution into each dispenser. The implant which is to receive the solution is placed on the support portion of the base unit, the nozzles of the dispensers are inserted into the hubs on the domed lid of the dosing chamber and the base and lid are placed in mating relationship and, if necessary or desirable, temporarily locked together with one or more releasable clasp(s) (not shown). The plungers are then moved downwardly to eject the solution from the dispensers into the communicating passageways in the lid and, if the volume of solution is greater than the volume of the associated passageway, onto the surface of the implant. The vacuum pump is then turned on to lower the pressure within the dosing chamber, thereby drawing out any solution from the passageways. Also, the design is such that the flow within the chamber is toward the perimeter of the implant, thus ensuring efficient transfer of solution over the surface of the implant toward its periphery. The vacuum pump is turned off when visual inspection confirms adequate transfer of solution onto the surface of the implant. The lid and base portions are then disassembled and the dosed implant is removed from the base support and placed in the patient.

An alternative delivery system may be designed without application of vacuum to the dosing chamber, assuming the volume of each passageway in the lid is rather precisely known. In this case, the volume of solution drawn into each dispenser through the adapter nozzle would be the sum of the volume of the respective passageway and the volume of fluid to b deposited on the implant. This assumes that each passageway is left filled with solution after the requisite amount of solution has been deposited on the implant.

What is claimed is:

1. A system for delivering a controlled volume of a solution carrying a bioactive agent and depositing the solution on a surface of a medical/dental implant in preparation for placing the implant in a patient, comprising:
a. at least one elongated dispenser each of which comprises a first nozzle positioned at the terminal end thereof, and a plunger positioned there-within for axial, reciprocating movement;
b. an adapter having a first surface with a first set of hubs positioned thereon, with the number of hubs in said first set of hubs being at least equal in number to the number of said at least one dispensers, and a second surface with a second nozzle extending downwardly therefrom, said second nozzle and said first set of hubs being in fluid communication with one another; and
c. a dosing chamber adapted to receive the implant therein and including a second set of hubs mounted on the exterior thereof and in fluid communication with the interior of said dosing chamber, wherein the number of hubs in said second set of hubs is at least equal to said number of said at least one dispensers.

2. The system according to claim 1, wherein said at least one elongated dispenser comprises at least two elongated dispensers each of which comprises a first nozzle at the terminal ends thereof, and a plunger positioned there-within for axial, reciprocating movement.

3. The system according to claim 2, further comprising a handle interconnected to each of said plungers and adapted to provide equal, simultaneous axial movement to each of said plungers.

4. The system according to claim 3, wherein said handle is associated with means for selective, calibrated movement.

5. The system according to claim 4, wherein said means for selective calibrated movement comprise a dosing gun with which said handle is associated, and a connector interconnecting said handle to each of said plungers.

6. The system according to claim 2, wherein said at least two elongated dispensers are arranged in a predetermined pattern.

7. The system according to claim 6, wherein each of said hubs in said first set of hubs is arranged in the same said predetermined pattern.

8. The system according to claim 6, wherein each of said hubs in said second set of hubs is arranged in the same said predetermined pattern.

9. The system according to claim 1, wherein said dosing chamber comprises a lid and a base removably connected to one another, wherein said second set of hubs are mounted to said lid and said lid and said base define an enclosed space when connected to one another.

10. The system according to claim 9, further comprising a vacuum port formed in said dosing chamber.

11. The system according to claim 9, wherein said lid is composed of a material that permits visual observance therethrough.

12. The system according to claim 9, further comprising a plate mounted within said enclosed space.

13. A method for delivering a controlled volume of a solution carrying a bioactive agent and depositing the solution on a surface of a medical/dental implant in preparation for placing the implant in a patient, comprising the steps of:
a. providing at least one dispenser each of which comprises a nozzle at the terminal end thereof and a plunger positioned therein that is capable of axial reciprocating movement;
b. providing an adapter having a first set of hubs and adapted to be used for filling each said nozzle with a predetermined volume of the solution;
c. mounting each said nozzle on a respective hub selected from said first set of hubs; and
d. filling each said nozzle with the solution by drawing the solution through said adapter and into each said nozzle.

14. The method according to claim 13, comprising the further steps of providing a dosing chamber.

15. The method according to claim 14, comprising the further steps of providing an implant plate, positioning the implant on said implant plate, and inserting the plate in said dosing chamber.

16. The method according to claim 15, comprising the further step of mounting each said nozzle to said dosing chamber, and dispensing the solution from said nozzle into said dosing chamber in coating relation to the implant.

17. A dosing chamber adapted for use in combination with a system that utilizes at least one nozzle for delivering a controlled volume of a solution carrying a bioactive agent and depositing the solution on a surface of a medical/dental implant in preparation for placing the implant in a patient, said dosing chamber comprising:
a. a lid;
b. a base to which said lid is releasably mounted and which collectively define an enclosed space when mounted to one another;
c. wherein said lid comprises at least one hub adapted to receive the at least one nozzle therein, said hub being in fluid communication with said enclosed space.

18. The dosing chamber according to claim 17, further comprising a vacuum port associated with said base.

19. The dosing chamber according to claim 17, further comprising a gasket sealingly positioned at the interface between said lid and said base.

20. A dispenser for use in combination with a system for delivering a controlled volume of a solution carrying a bioactive agent and depositing the solution on a surface of a medical/dental implant that is positioned within a dosing chamber, in preparation for placing the implant in a patient, said dispenser comprising:
a. at least two elongated cylinders each of which includes a nozzle extending from the terminal ends thereof;
b. at least two plunger members each one of which is mounted in a respective one of said at least two elongated cylinders for selective, axial movement therein; and
c. a handle interconnected to each of said at least two plunger members and adapted to provide equal, simultaneous axial movement to each of said at least two plungers.

21. The dispenser according to claim 20, wherein said handle is associated with means for selective, calibrated movement.

22. The system according to claim 21, wherein said means for selective calibrated movement comprise a dosing gun with which said handle is associated, and a connector interconnecting said handle to each of said plungers.

23. An adapter for use in combination with a system for delivering via at least two first nozzles a controlled volume of a solution carrying a bioactive agent and depositing the solution on a surface of a medical/dental implant that is positioned within a dosing chamber, in preparation for placing the implant in a patient, said adapter comprising:
a. a plate having first and second opposing surfaces;
b. at least two hubs positioned on said first surface and adapted to receive the at least two first nozzles; and
c. a second nozzle positioned on said second surface, wherein said second nozzle and said at least two hubs are in fluid communication with each other.

24. A system for delivering a controlled volume of a solution carrying a bioactive agent and depositing the solution on a surface of a medical/dental implant in preparation for placing the implant in a patient, comprising:
a. at least one elongated dispenser;
b. an adapter having a first surface adapted to be a fluidly connected to said at least one elongated dispenser, and a second surface that is in fluid communication with said first surface; and
c. a dosing chamber defining an enclosed space that is adapted to receive the implant therein, wherein said at least one dispenser is removably positionable on said dosing chamber such that it is in fluid communication with said enclosed space.

25. The system according to claim 24, wherein each of said at least one dispenser comprise a first nozzle positioned at the terminal end thereof, and a plunger positioned there-within for axial, reciprocating movement.

26. The system according to claim 25, wherein said adapter comprises an upper surface with a first set of hubs positioned thereon, with the number of hubs in said first set of hubs being at least equal in number to the number of said at least one dispensers, and a lower surface with a second nozzle extending downwardly therefrom, said second nozzle and said first set of hubs being in fluid communication with one another.
